(19) United States
(12) Patent Application Publication
(10) Pub. No.: US 2002/0186851 A1
(43) Pub. Date: Dec. 12, 2002

(54) International battery holder for use with a electronic stethoscope

(76) Inventors: Richard J. Deslauriers, Woodbury, CT (US); Robert T. Potash, S. Windsor, CT (US)

Correspondence Address: RICHARD J. DESLAURIERS, 143 WOLCOTT ROAD, WOLCOTT, CT, 06716, US

(21) Appl. No.: 09/877,194

(22) Filed: Jun. 11, 2001

(51) Int. Cl. A61B 7/04

(52) U.S. Cl. 381/67

(57) ABSTRACT

The disclosed invention relates to a method for the holding of a power source, such as a battery, within the head of a digital stethoscope in order to provide power to electronic and digital features offered by certain electronic stethoscopes. This disclosed battery holder teaches a method of incorporating a battery, or other portable power source, into the head of a stethoscope without compromising the conventional stethoscope's advantages of size, convenience, and ease of use. Additionally, The disclosure provides for a device for holding a battery cell that allows for easy replacement of the battery and does not encompass the stethoscope head so as inhibit the use of other features.

### CROSS-REFERENCES TO RELATED APPLICATIONS

### STATEMENT REGARDING FEDERALLY SPONSORED REESEARCH OR DEVELOPMENT

### REFERENCE TO A MICROFICHE APPENDIX

### BACKGROUND OF THE INVENTION

1. Field of the Invention

This invention relates to the field of medical devices, and in particular a method of incorporating a battery into stethoscopes or similar auscultation equipment.

2. Description of the Related Art

The stethoscope is comprised of a stethoscope head, tubing or lumens, the stethoscope binurals, and the ear tips. The conventional stethoscope head is designed such that its size allows for ease of movement, storage, and certain acoustic properties. Whereas due to the limited space available on the stethoscope head, the incorporation of batteries and other electronic devices and features to a stethoscope while retaining the function and appearance of a conventional stethoscope, but enhancing the conventional stethoscope with digital features, has proven unsuccessful in the prior art. One particular problem arising in the placement of a power source, such as batteries, within a conventional stethoscope body is to construct the proper type of power source that fits within the same geometry as a conventional stethoscope and is able to be placed in the sound traveling path for the stethoscope. Furthermore, the incorporation of a battery must be designed in order not to affect the ability of the stethoscope to capture a desired sound and carry that sound acoustically through the stethoscope head assembly without hampering the sound being transmitted. Another problem is the need find a location within the stethoscope body that allows easy access in order to change the power source when the power source has failed without the need for additional tooling.

This invention discloses a device for holding a power source, such as a battery, within the stethoscope head assembly of a digital stethoscope such that the stethoscope head retains the same appearance as a conventional stethoscope, and such as to affix a power source such as not to adversely affect the quality or amplitude of the captured sound source. This invention further discloses a device that allows the user to have easy access to the power source to facilitate changing from time to time.

### BRIEF SUMMARY OF THE INVENTION

According to the first objective of the present invention, a battery holder for use in a conventional, designed stethoscope head having an electronic assembly is hereby disclosed, which invention permits the holding of one or more batteries, so as to provide power to electronic or digital functions of an electronic stethoscope, and whereby the incorporation of said battery holder in a stethoscope such that the path in which sound is traveling in the stethoscope travels around this disclosed battery holder.

Another objective of the present invention is to teach a battery holder for use in an electronic stethoscope head that incorporates one or more air passages so as not to disrupt the ability of the stethoscope to transmit sound through and around the power source and diminish the quality of sound that is produced.

In the preferred embodiment of the present invention, a battery holder is disclosed, whereby, this battery holder is manufactured of an inexpensive plastic having a specific diameter, correlating to the diameter of a stethoscope's head, and whereby one or more sets of air holes are manufactured into the base of this battery holder, so as to permit sound to travel around the batteries and through this batter holder into the stethoscope lumen(s).

Furthermore, in this inventions preferred embodiment, a battery holder for an electronic stethoscope is disclosed that permits the attachment of the control board for the electronic stethoscope and certain electronic connections, such as positive and negative wireless electrical conduits for transferring power generated by the power source such that power from the stored batteries is transmitted to the control board of a digital stethoscope without the use of additional wires conducting power from the batteries to the control board.

In another preferred embodiment, a battery holder is disclosed for use in a stethoscope that allows for simplified removal and replacement of the batteries, the battery holder, or the control board as needed.

### BRIEF DESCRIPTION OF THE SEVERAL VIEWS

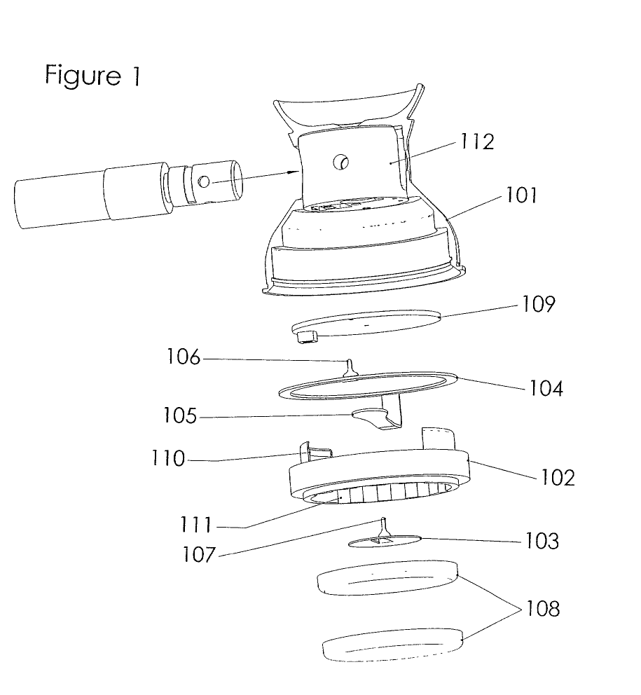

FIG. 1 depicts an exploded side-view of an electronic stethoscope head-piece.

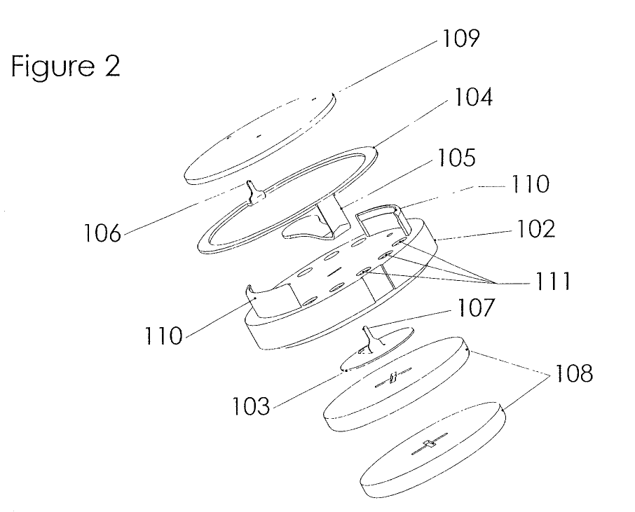

FIG. 2 depicts an exploded side-view of the battery holder.

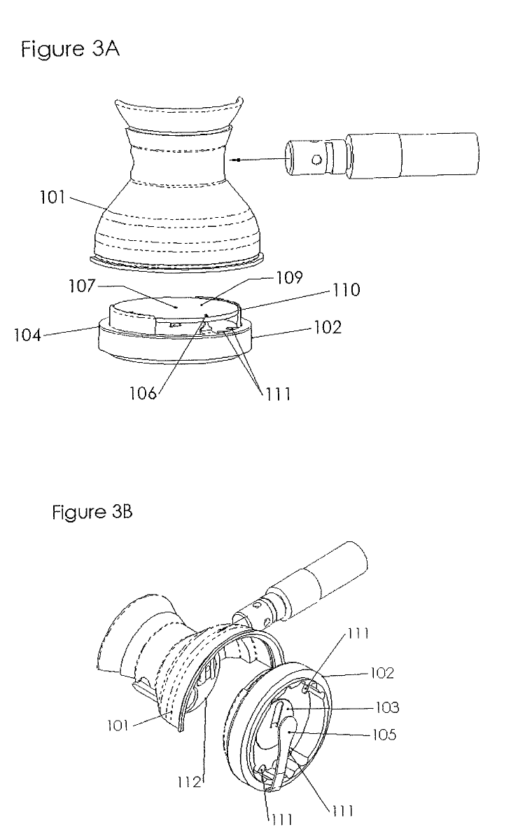

FIG. 3a depicts a three-dimensional side-view of this battery holder and stethoscope head from the top.

FIG. 3b shows a three-dimensional side-view of this battery holder with a stethoscope head from the bottom.

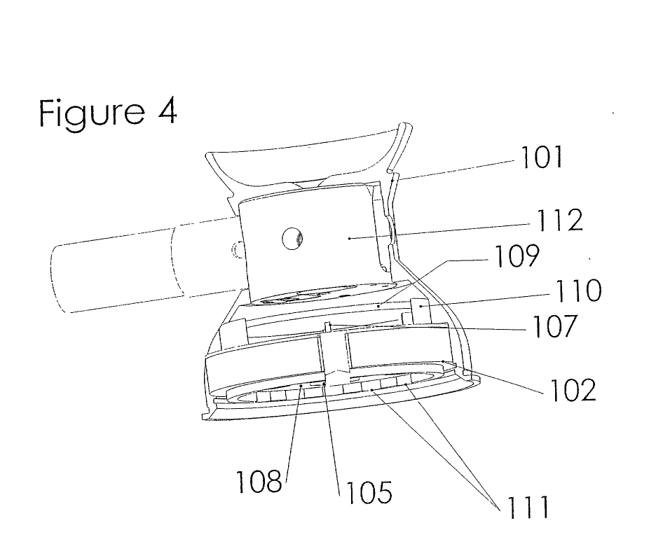

FIG. 4 depicts a side-view sectional drawing of a stethoscope utilizing this invention.

### DETAILED DESCRIPTION OF THE SEVERAL VIEWS

Whereby in referring to the forthcoming FIGS. 1-4 in connection with this disclosed Specification, like reference numbers appearing on separate Figures refer to like parts of this disclosed invention.

Referring to FIG. 1, an exploded view of the preferred embodiment of the present invention in connection with its placement in an electronic stethoscope. A stethoscope Shell 101 is shown having a Control Spool 112 located in the center of said Shell 101. Further shown are the components that comprise this invention including Battery Holder 102 incorporating Flanges 110 and Air Pathways 111. Other elements comprising the present invention include Retaining Ring 104 with Negative Battery Contact (NBC) 105 and Negative Control Board Contact (NCBC) 106, Positive Battery Contact (PBC) 103 with Positive Control Board Contact (PCBC) 107, Batteries 108, and Control Board 109.

Further illustrated in FIG. 2 is an exploded three-dimensional view of the components of this invention and the arrangement of its preferred embodiments. Battery Holder 102 is a manufactured plastic or similar resin that has a diameter no greater than the diameter of the shell of the stethoscope, such that Battery Holder 102 fits securely within the stethoscope shell, and further whereby Battery Holder 102 has a recess for the placement of Batteries 108. Additionally, Battery Holder 102 incorporates Flanges 110, whereas such Flanges 110 are manufactured with a groove in which Control Board 109 is to be secured. This Battery Holder 102 further incorporates one or more Air Pathways 111, whereby pursuant to these Air Pathways 111, sound energy is permitted to pass through these Air Pathways 111 when the stethoscope is used in capturing sound resonations from within a patient's body, and whereby these Air Pathway's 111 allow the battery holder to be placed in the line of the sound resonations being captured by the stethoscope head with negatively affecting the acoustic characteristics of the stethoscope.

PBC 103 is an electrically conducting material, such as brass, and has a protruding PCBC 107, whereby PBC 103 is placed in the inside of Battery Holder 102 with PCBC 107 being inserted through a hole in Battery Holder 102, and thereby creating a wireless contact for providing power to Control Board 109 when Control Board 109 is secured within Flanges 110. Retaining Ring 104 is manufactured to have an opening in the middle with a diameter slightly larger the diameter caused from Flanges 110 and whereby said diameter of Retaining Ring 104 is no larger than the diameter of Battery Holder 102, and thereby permitting Retaining Ring 104 to fit snuggly around and on top of Battery Holder 102. Furthermore, Retaining Ring 104 also has NBC 105 that extends from the outer rim of Retaining Ring 104 over and underneath Battery Holder 102, such that Retaining Ring 104 is secured to Battery Holder 102 after Batteries 108 are inserted and hold in place NBC 105, and whereas Batteries 108 are similarly held within Battery Holder 102 through the use of NBC 105. NCBC 106 is a protruding member of Retaining Ring 104 and when Retaining Ring 104 is secured in place around Battery Holder 102, then NCBC 106 acts to complete the power circuit used to conduct power supplied from Batteries 108 to Control Board 109.

FIG. 3a depicts the disclosed invention in its preferred embodiment as the components interconnect forming this disclosed battery holder for use in a conventionally designed electronic stethoscope. Specifically shown in FIG. 3 are the interconnections between Control Board 109 and Battery Holder 102. As seen, Control Board 109 snaps into Flanges 110. Control Board 109 as designed for this invention accommodates wireless battery contacts, such that when Control Board 109 is secured within Flanges 110, positive and negative anodes which directly contact the PCBC 107 and NCBC 106 respectively without the need for any additional power wires. Additionally, the diameter of Control Board 109 is smaller than the diameter of the Battery Holder 102 thereby preventing the Control Board from becoming an impediment to the passage of sound waves through Shell 101, and such that the angle so formed from the Flanges 110 and Battery Holder 102 follows the contour of said Shell 101 to facilitate assembled Battery Holder 102 placement therein.

FIG. 3b shows the assembled battery holder being comprised of its preferred embodiments. As demonstrated in this Figure and explained in the aforementioned paragraphs from a underneath perspective, whereas Air Pathways 111 are seen in the base of Battery Holder 102, and also shown in this FIG. 3b are PBC 103 and NBC 105 as located in their preferred positioning within Battery Holder 102.

FIG. 4 depicts the preferred embodiments of the present Battery Holder 102 in its intended position within the Shell 101, and whereas reference numbers refer to the preferred embodiments as aforementioned in the previous paragraphs.

What is claimed:

1. A device for securing batteries whereby said device is characterized as a battery holder whereas when manufactured said battery holder is to be inserted into a stethoscope head assembly of a specified size, whereby a shallow cavity is manufactured within a stethoscope head assembly, and whereas said shallow cavity encloses the disclosed battery holder, a means of securing within the stethoscope head assembly so as to provide an internal power source powering digital features to a conventional stethoscope design, whereby,
said device is incorporated into the existing shape of a conventional stethoscope head whereby the stethoscope head retains the same overall shape as a conventional stethoscope head, through the use of a battery holder which permits air to travel through said battery holder, through one or more holes placed though said battery holder, and therefore not impeding the transmission of sound energy through the stethoscope head cavity.

2. A battery holder disclosed in claim 1, whereas the battery holder retains a electronic or digital operations control board and utilizes certain electronic connections as to transfer power from the housed batteries to and enabling said control board.

3. A battery holder, whereas the battery holder is located within the shell of a stethoscope or the acoustic chamber in the direct path of any sound energy traveling through said acoustic chamber, of which battery holder is sized to retain the functional and aesthetic appearance of the standard stethoscope shell.

4. A battery holder as disclosed in claim 3, whereas the battery holder is adapted to utilize conventional coin shaped batteries that maximize power as related to the size and shape of said batteries.

5. A battery holder as disclosed in claim 3, whereas the battery holder is adapted and sized to utilize any convention battery source.

6. A battery holder as disclosed in claim 3, whereas the battery holder is adapted and sized to utilize any custom designed battery source.

7. A battery holder and batteries secured within said acoustic chamber such that the batteries are retained through a manually attachable element and positioned directly under the hard or soft diaphragm of a stethoscope, therefore requiring no special needs or tools for accessing the enclosed batteries.
